(19) United States
(12) Patent Application Publication
(10) Pub. No.: US 2010/0267818 A1
(43) Pub. Date: Oct. 21, 2010

(54) STABILIZED SINGLE-LIQUID PHARMACEUTICAL COMPOSITION CONTAINING DOCETAXEL

(75) Inventors: Moo-Hi Yoo, Seoul (KR); Bong-Jin Cha, Anyang-si (KR); Jeong-Hoon Kim, Seongnam-si (KR); Sun-Woo Jang, Seoul (KR); Dong-Han Won, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: DONG-A PHARM. CO., LTD, Seoul (KR)

(21) Appl. No.: 12/826,278

(22) Filed: Jun. 29, 2010

Related U.S. Application Data
(63) Continuation of application No. PCT/KR2009/000911, filed on Feb. 26, 2009.

(30) Foreign Application Priority Data
Feb. 29, 2008 (KR) 10-2008-0019179

(51) Int. Cl. A61K 31/337; A61P 35/00

(52) U.S. Cl. 514/449

(57) ABSTRACT

This invention relates to a single-liquid pharmaceutical composition for injection containing docetaxel. The composition includes (A) docetaxel and pharmaceutically acceptable salts thereof, (B) a surfactant selected from the group consisting of polysorbate, polyoxyethylene glycol ester and polyoxyethylene castor oil derivatives, (C) a solvent comprising anhydrous ethanol in a concentration range of 100 to 800 mg/ml, in an injectable solution, and (D) a pH adjuster of an amount suitable for adjusting the pH of the liquid composition to 5 or less. The composition may be directly diluted in a perfusion liquid even without the use of an intermediary dilute solution in case the composition is used for injectable preparations since the composition is in a single liquid phase. Furthermore, the composition is suitable for effective administration of docetaxel since the pharmaceutical stability of the composition is significantly improved.

### TECHNICAL FIELD

The present invention relates to a stabilized single-liquid pharmaceutical composition for injection comprising docetaxel.

### BACKGROUND ART

Docetaxel is one of semi-synthetic toxoid derivatives that have been used as an anti-cancer drug. The formula of docetaxel is presented by 4-acetoxy-2α-benzoyloxy-5β,2O-epoxy-1,7β,10β-trihydroxy-9-oxo-tax-11-en-13β-yl(2R,3 S)-3-tert-butoxycarbonylamino-2-hydroxy-3-phenyl propionate, and its chemical structure is represented by the following formula 1. The empirical formula of docetaxel is represented by C₄₃H₅₃NO₁₄, which is in the form of white or whitish powder, and it was known that the docetaxel is a highly liposoluble and poorly water-soluble drug with water solubility of 6 to 7 μg/mL.

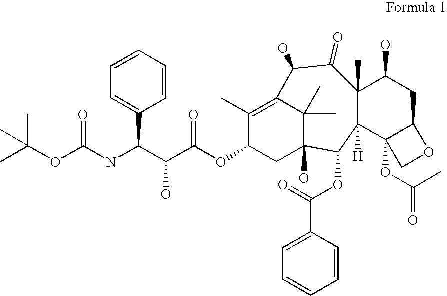

Korean Patent Registration No. 136722 discloses a composition that is suitable for injections and substantially free of ethanol, and includes water-insoluble taxane derivatives which are dissolved in a surfactant selected from the group consisting of polysorbate, polyoxyethylene glycol ester and polyoxyethylene castor oil derivatives.

In recent years, docetaxel was commercially available from Aventis, which is named Taxotere®, and its technical basis relies on Korean Patent Registration No. 136722. In accordance with the registered patent, it is necessary to administer an effective component in a suitable amount for the treatment of cancers, but patients may be exposed to the risks of anaphylactic shock or alcoholism which may appear during the injection therapy since the effective component is used in a large amount due to its low concentration in an injectable solution. Therefore, the registered patent describes that it is necessary to substantially completely remove ethanol which is included during the process of preparing a docetaxel-containing injectable composition.

However, the commercially available Taxotere® should be used by mixing the two components—a 13% ethanol-diluted solution and a concentrated docetaxel solution to prepare a Pre-mix solution. The mixed solution contains a docetaxel concentration of 10 mg/ml, and is generally taken with a scaled syringe in a suitable amount and injected into a 250 ml sap bag or vial containing 0.9% physiological saline or a 5% glucose solution. When the docetaxel is necessarily used in an amount greater than 200 mg, it is recommended to use the docetaxel in a final dilution concentration of no more than 0.74 mg/ml.

In the case of the administration of Taxotere®, it is true to administer an ethanol-containing solution for injection, but there is no reports on the risks of anaphylactic shock or alcoholism caused by the ethanol content during the treatment with a large amount of injection in the actual clinical trials.

Therefore, the content of ethanol used as a solvent for injections in the docetaxel-containing injectable preparation does not affect the safety of the injectable preparation, unlike the description of the prior-art literatures.

Recently commercially available Taxotere® has problems in that the drug administration is very complicated, which includes: primarily mixing a drug concentrate with a dilute solution to prepare a pre-mix solution, secondarily diluting the pre-mix solution with 0.9% physiological saline, and instilling the resultant pre-mix dilution for approximately 1 hour within 4 hours after the preparation of the pre-mix dilution. Also, the Taxotere® has demerits in that the drug concentrate mixed with the dilute solution should be carefully turned upside down for 45 seconds to prepare a pre-mix solution without stirring, and bubbles may be formed in the obtained solution, and the solution should be kept for 5 minutes to get rid of the bubbles. Since the pre-mix solution prepared thus is stably stored only for 8 hours at a temperature of 2 to 8° C. or a room temperature, the pre-mix solution should be diluted with a perfusion liquid.

As described above, the docetaxel preparation, Taxotere®, whose technical basis relies on Korean Patent Registration No. 136722 is formed into a gelled phase when it is directly diluted with perfusion liquid. Therefore, the docetaxel preparation has a problem in that the cumbersome and inconvenient procedures, such as the primary dilution with 13% ethanol dilute solution and the re-delution with perfusion liquid, are required for the use of Taxotere® to be administered. In this case, the primarily diluted solution has a problem in that it is stored for a limited period of 8 hours at room temperature or refrigerative temperature due to its low stability.

Korean Patent Registration No. 607391 discloses a water-soluble solid pharmaceutical composition and aqueous solutions thereof, comprising docetaxel and cyclodextrin, wherein the docetaxel is used for the parenteral preparations. However, the prior-art patent has defects in that since a weight ratio of docetaxel and cyclodextrin used is in a range greater than 1:50, the expensive cyclodextrin is used in a large amount, which leads to a significant increase in the production costs. Also, it has a problem in that the manufacturing process is very complicated since the docetaxel-containing water-soluble solid should be prepared through the dry-freezing procedure.

Meanwhile, Korean Patent Registration No. 330316 discloses an injectable composition comprising taxane derivatives and a surfactant selected from the group consisting of polysorbates, ester-ether of ethylene oxides, and ester-ether of fatty acid glycerides, wherein the injectable composition has two compartments which are used to prepare a solution containing less than 5% by weight of ethanol and an injectable solution including taxane derivatives in which a diluent selected from the group consisting of organic compounds and sodium chloride is present in an amount of 6% by weight based on the total amount of the surfactant, the organic compounds being able to prevent the formation of a gelled phase or disintegrate the gelled phase while the ethanol-containing solution is mixed with an aqueous solution and the organic compounds containing a hydroxyl group or an amine functional group and having a molecular weight of less than 200. Also International Publication No. WO 06/133510 describes a liquid composition for injection comprising docetaxel, polyethylene glycols, and an insoluble solvent, wherein the pH of the composition is in a range of pH 2.5 to 7. However, such a liquid composition is unstable, that is, the content of related compounds may be substantially increased during an accelerated 1-month storage test, as describe in Comparative examples of the specification of this application, and precipitates may be formed when the composition is diluted with a secondary perfusion liquid and kept for an extended time of period.

In addition, a variety of researches regarding the docetaxel have been made in the art. For example, Korean Patent Registration No. 401119 discloses a pharmaceutical composition comprising unsaturated phospholipids and a small amount of other negative phospholipids, wherein the taxoid-based active ingredient is stable and highly concentrated, and US Patent Publication No. 2006-188566 discloses a method for preparing docetaxel nanoparticles containing a surface stabilizing agent. Also, US Patent Publication No. 2006-67952 discloses a method for preparing a docetaxel o/w emulsion for injection using a low-capacity oil, and US Patent Publication No. 2007-82838 discloses a technology using a stabilizing agent such as serum albumin so as to improve the stability of a nanoparticle suspension containing a poorly water-soluble drug such as docetaxel when diluted prior to the administration.

Docetaxel is a drug that is highly toxic and used in a very small amount. Therefore, in order to safely administer the docetaxel, an injectable preparation is necessarily prepared, which may be readily handled and be suitable for administering an exact amount of the docetaxel.

The present inventors have made attempts to improve the stability of an injectable preparation comprising docetaxel and develop a method for administering the same, and found the effects of a single-liquid injectable pharmaceutical composition comprising docetaxel, which may be directly used without the use of an intermediary dilute solution due to its highly improved long-term storage stability. Therefore, the present invention is perfected on the basis of the above-mentioned facts.

### DISCLOSURE

### Technical Problem

Accordingly, the present invention is designed to solve such drawbacks of the prior art, and therefore an object of the present invention is to provide a single-liquid composition for injection containing docetaxel, which is able to be directly used without the use of an intermediary dilute solution by improving the stability of an injectable preparation containing docetaxel and developing a method for administering the injectable preparation.

### Technical Solution

Hereinafter, exemplary embodiments of the present invention will be described in detail.

In order to solve the problems of the prior art, the single-liquid docetaxel injectable composition should satisfy the following requirements:

First, poorly water-soluble docetaxel should be dissolved

Second, the stability of the composition in which the docetaxel is dissolved should be secured, and

Third, the injectable composition should have excellent dilution stability during the dilution with a perfusion liquid.

According to an aspect of the present invention, there is provided a single-liquid pharmaceutical composition for injection containing docetaxel, including (A) docetaxel and pharmaceutically acceptable salts thereof; (B) a surfactant selected from the group consisting of polysorbate, polyoxyethylene glycol ester and polyoxyethylene castor oil derivatives; (C) a solvent including anhydrous ethanol in a concentration range of 100 to 800 mg/ml in an injectable solution, and (D) a pH adjuster of an amount suitable for adjusting the pH of the liquid composition to 5 or less.

The single-liquid docetaxel-containing pharmaceutical composition according to the present invention is easy to be administered and show more excellent stability than a pre-mix solution in which a drug concentrate is mixed with a dilute solution since the pharmaceutical composition may be directly diluted and used without the use of an intermediary dilute solution. In particular, the pharmaceutical composition has a significantly improved pharmaceutical stability, as well as the excellent long-term storage stability since the disintegration of drugs may be prevented at the presence of ethanol, thereby providing more various merits than the conventional preparations. Also, since the conventional preparations are composed of a high-viscosity surfactant, they cannot be administered dividedly. However, as the mixture of ethanol and a surfactant, the pharmaceutical composition according to the present invention may be administered dividedly since it has low viscosity.

The docetaxel in the composition of the present invention includes all types such as anhydrides, hydrates, polymorphs, derivatives and prodrugs. As the anhydride, the docetaxel in the composition is present in a concentration of 5 to 80 mg/ml, and preferably 10 to 30 mg/ml.

The surfactant used as a solubilizing agent according to one exemplary embodiment of the present invention is selected from the group consisting of polysorbates such as Tween80®, polyoxyethylene glycol esters such as Emulphor®, and polyoxyethylene castor oil derivatives such as Cremophore ELP®, etc.

Also, the solvent according to one exemplary embodiment of the present invention includes anhydrous ethanol, and may be used to dissolve docetaxel and reduce the viscosity of the final preparation.

In particular, it is impossible to dividedly administer the conventional preparations since they are composed of a high-viscosity surfactant, but the composition according to the present invention as the mixture of ethanol and a surfactant may be administered dividedly since it has a low viscosity. When the composition is diluted with a perfusion liquid for the purpose of the administration to patients, the addition of ethanol facilitates the dilution of the composition since the ethanol functions to prevent gelation. However, since the use of excess ethanol may cause the disintegration of drugs and the symptoms of alcoholism, the ethanol used should be adjusted to a proper dose. The solvent in the composition of the present invention is present in a concentration of 100 to 800 mg/ml. Here, when a solvent is present in a lower concentration than the solvent according to one exemplary embodiment of the present invention, the solvent is allowed to deteriorate the dilution stability, but the higher concentration of the solvent may cause the disintegration of drugs and the symptoms of alcoholism.

The pH adjuster according to one exemplary embodiment of the present invention is selected from the group consisting of citric acid, fumaric acid, lactic acid, stannic acid, succinic acid, maleic acid, acetic acid, tartaric acid, oxalic acid, phosphoric acid, and hydrochloric acid, and citric acid may be preferably selected as the pH adjuster.

According to the present invention, when docetaxel is dissolved with polysorbate in anhydrous ethanol, the pH of the composition is in a range of approximately 7.0, and the content of the docetaxel may be significantly lowered and the content of its related compounds may be increased over a 15-day storage period at room temperature or an accelerated 15-day storage period. Therefore, the pH of the composition of the present invention should be essentially adjusted to 5 or less, preferably 3 to 5, by using a pH adjuster.

The composition of the present invention may be very easily prepared through the following steps.

1) Docetaxel is dissolved in anhydrous ethanol, and completely dissolved by the addition of a pH adjuster. 2) The resultant solution is mixed and stirred while a solubilizing agent is slowly added to the solution until the homogenous solution is obtained. 3) The resultant mixture is sterilely filtered and put into glass vials.

### Advantageous Effects

As described above, the single-liquid pharmaceutical composition for injection according to the present invention is an injectable preparation that may prevent the disintegration of drugs even when the docetaxel is manufactured into the pharmaceutical composition including ethanol, and also shows its excellent long-term pharmaceutical storage stability. Also, the pharmaceutical composition is easy to be administered since it may be directly diluted and used without the use of an intermediary dilute solution.

### Best Mode

Hereinafter, exemplary embodiment of the present invention will be described in detail.

However, it should be understood that the description proposed herein is just a preferable example for the purpose of illustrations only, not intended to limit the scope of the invention.

### EXAMPLES 1 to 8

### Preparation of Inventive Docetaxel-Containing Liquid Preparations

200 mg of docetaxel (anhydrous) was completely dissolved in anhydrous ethanol, based on the compositions and contents as listed in the following Table 1, and acid was further added and dissolved. Then, the resultant solution was mixed and stirred while a solubilizing agent is slowly added to the solution until the homogenous solution was obtained. The final solution was filtered through a 0.22 μm filter and put into glass vials to prepare a single-liquid docetaxel-containing injection, which was used in Examples 1 to 8, respectively.

TABLE 1

| Unit: mg | Example 1 | Example 2 | Example 3 | Example 4 | Example 5 | Example 6 | Example 7 | Example 8 |
|---|---|---|---|---|---|---|---|---|
| Docetaxel (anhydrous) | 200 | 200 | 200 | 200 | 200 | 200 | 200 | 200 |
| Polysorbate 80 | 5200 | — | 5200 | 5200 | 5200 | 5200 | 5200 | 5200 |
| (Tween 80 ®) | | | | | | | | |
| Polyoxyl 35 castor oil | — | 5200 | — | — | — | — | — | — |
| (Cremophore ELP ®) | | | | | | | | |
| Anhydrous ethanol | 3900 | 3900 | 1910 | 8000 | 3900 | 3900 | 3900 | 3900 |
| Citric acid | 20 | 20 | 20 | 20 | — | — | 10 | 30 |
| Acetic acid | — | — | — | — | 20 | — | — | — |
| Lactic acid | — | — | — | — | — | 20 | — | — |

### Comparative examples 1 to 9

### Preparation of Docetaxel Preparations

Docetaxel preparations were prepared based on the compositions and contents as listed in the following Table 2, and were used in Comparative examples 1 to 9, respectively.

In particular, a commercially available product, Taxotere® liquid, was used in Comparative example 2, and it was prepared, as described in Korean Patent Registration No. 136722, by dissolving docetaxel in anhydrous ethanol and putting polysorbate 80 into the resultant solution and evaporating the anhydrous ethanol at 30° C. for 2 hours in a rotary evaporator.

TABLE 2

| | Comp. | Comp. | Comp. | Comp. | Comp. | Comp. | Comp. | Comp. | Comp. |
|---|---|---|---|---|---|---|---|---|---|
| Unit: mg | Ex. 1 | Ex. 2 | Ex. 3 | Ex. 4 | Ex. 5 | Ex. 6 | Ex. 7 | Ex. 8 | Ex. 9 |
| Docetaxel (anhydrous) | 200 | 200 | 200 | 200 | 200 | 200 | 200 | 200 | 200 |
| Polysorbate 80 | — | 5200 | 5200 | 5200 | 5200 | 5200 | 5200 | 5200 | 5200 |
| (Tween 80 ®) | | | | | | | | | |
| Polyoxyl 35 castor oil | — | — | — | — | — | — | — | — | — |
| (Cremophore ELP ®) | | | | | | | | | |
| Macrogol 15 | 5200 | — | — | — | — | — | — | — | — |
| Hyroxystearate | | | | | | | | | |
| (Solutol HS ®) | | | | | | | | | |
| PEG300 | — | — | 3900 | — | — | — | — | — | — |
| PEG400 | — | — | — | 3900 | — | 3900 | — | — | — |
| Pharmasolv | — | — | — | — | 3900 | — | — | — | — |
| Anhydrous ethanol | 3900 | — | — | — | — | 3900 | 1900 | 3900 | 3900 |
| Citric acid | 20 | — | 20 | 20 | 20 | 20 | — | — | 5 |
| Acetic acid | — | — | — | — | — | — | — | — | — |
| Lactic acid | — | — | — | — | — | — | — | — | — |

### Experimental example 1

### Effects of Surfactant in Inventive Docetaxel Composition

The effects of the surfactant on the stability of the docetaxel-containing injections prepared in Examples 1 and 2 and Comparative example 1 were evaluated at room temperature and under the accelerated storage conditions (40° C. and 75% RH).

The content of docetaxel in the prepared solutions and its related compounds were analyzed at the following conditions by using a HPLC system.

1) Mobile phase—0.02M sodium acetate buffer (adjusted to pH 4.5): acetonitrile=60:40

2) Column—Hypersil MOS 15 cm×4.6 mm, 5 μm, C8 or its equivalent column

3) Wavelength—230 nm

4) Flow rate—1.0 ml/min

5) Input—20 μl

In order to evaluate the content of the docetaxel, 10 mg of docetaxel (anhydrous) as a test sample was taken, and put into a 100-ml volumetric flask, dissolved in 20 ml in of acetonitrile, and then quantified into a mobile phase, which was used as the test sample. Also, a standard docetaxel (anhydrous) was taken at an exact amount of 10 mg, and prepared in the same manner as in the test sample. The resultant solution was used as the standard solution. In order to analyze the related compounds, 10 mg of docetaxel (anhydrous) as a test sample was taken, and put into a 10-ml volumetric flask, dissolved in 2 ml a of acetonitrile, and then quantified into a mobile phase, which was used as the test sample.

The pH of the prepared solutions was measured by diluting an equivalent amount (10 mg) of docetaxel (anhydrous) with 5 ml of an injectable solution, and their shapes were observed with the naked eye, depending on the changes in colors and the precipitation. The results are listed in the following Table 3.

TABLE 3

| | Stability at the beginning | | | | Accelerated 1-month storage stability | | | |
|---|---|---|---|---|---|---|---|---|
| | | | | Total related | | | | Total related |
| | | | Content | compounds | | | Content | compounds |
| | Shape | pH | (%) | (%) | Shape | pH | (%) | (%) |
| Ex. 1 | Colorless | 3.81 | 100.5 | 0.705 | Colorless | 3.91 | 100.4 | 1.104 |
| | and clear | | | | and clear | | | |
| Ex. 2 | Colorless | 3.54 | 101.4 | 0.715 | Colorless | 3.56 | 101.0 | 1.206 |
| | and clear | | | | and clear | | | |
| Comp. | Colorless | 4.22 | 100.7 | 0.865 | Light | 4.25 | 91.4 | 9.412 |
| Ex. 3 | and clear | | | | yellow | | | |

As listed in Table 3, it was revealed that the docetaxel-containing preparations of Examples 1 and 2 in which polysorbate 80 and polyoxyl 35 castor oil were used as the surfactant according to on exemplary embodiment of the present invention were all stable at room temperature and under an accelerated storage condition, but when solutol HS was used as the surfactant as described in Comparative example 1, the changes in shape and a decrease in content were observed under an accelerated 1-month storage condition.

Therefore, it was seen that the surfactant selected from the group consisting of polysorbates, polyoxyethylene glycol esters and polyoxyethylene castor oil derivatives is an important factor to secure the stability of the composition in which docetaxel is dissolved in the present invention in the case of the single-liquid docetaxel-containing pharmaceutical composition for injection.

### Experimental example 2

### Effect of Solvent in Inventive Docetaxel Composition

The effects of the docetaxel-containing injectable preparations according to the presence of the solvent, ethanol, were evaluated from the compositions prepared in Examples 1, 3 and 4 and Comparative examples 2 to 6.

The pH and shapes of the prepared solutions were measured in the same manner as in Experimental example 1. The results are listed in the following Table 4.

TABLE 4

| | Stability at the beginning | | | | Accelerated 1-month storage stability | | | |
|---|---|---|---|---|---|---|---|---|
| | | | | Total related | | | | Total related |
| | | | Content | compounds | | | Content | compounds |
| | Shape | pH | (%) | (%) | Shape | pH | (%) | (%) |
| Ex. 1 | Colorless | 3.81 | 100.5 | 0.705 | Colorless | 3.91 | 100.4 | 1.104 |
| | and clear | | | | and clear | | | |
| Ex. 3 | Colorless | 3.75 | 99.62 | 0.726 | Colorless | 3.76 | 100.09 | 1.290 |
| | and clear | | | | and clear | | | |
| Ex. 4 | Colorless | 3.82 | 100.12 | 0.711 | Colorless | 3.81 | 100.44 | 1.030 |
| | and clear | | | | and clear | | | |
| Comp. | Colorless | 6.90 | 101.1 | 0.680 | Light | 6.92 | 42.4 | 30.66 |
| Ex. 2 | and clear | | | | yellow | | | |
| Comp. | Colorless | 3.92 | 100.30 | 0.715 | Colorless | 3.85 | 100.25 | 1.765 |
| Ex. 3 | and clear | | | | and clear | | | |
| Comp. | Colorless | 3.84 | 99.84 | 0.695 | Colorless | 3.82 | 100.10 | 1.722 |
| Ex. 4 | and clear | | | | and clear | | | |
| Comp. | Colorless | 3.77 | 101.02 | 0.725 | Colorless | 3.80 | 98.16 | 1.744 |
| Ex. 5 | and clear | | | | and clear | | | |

According to the present invention, it was seen that the docetaxel-containing preparations of Examples 1, 3 and 4 were all stable in the accelerated 1-month storage test without the changes in the shape, the content and the kind of the related compounds when the anhydrous ethanol was in a concentration of 100 to 800 mg/ml in each injectable solution. Also, it was revealed that the docetaxel-containing preparation of Comparative example 2 in which the anhydrous ethanol and the pH adjuster are not used was unstable in the accelerated 1-month storage test, that is, its color was changed into light yellow, and the changes in the content of docetaxel and its related compounds were observed, as well.

Also, it was seen that the docetaxel-containing preparations of Comparative examples 3 to 5 in which glycols are used instead of the anhydrous ethanol as the solvent and a pH adjuster is also used were stable at the beginning and under the accelerated 1-month storage condition since the changes in the contents and the total related compounds were inferior to those of Examples 1, 3 and 4 of the present invention.

Therefore, it was seen that, when the anhydrous ethanol was in a concentration of 100 to 800 mg/ml in each injectable solution, it is an important factor to secure the stability of the composition in which docetaxel is dissolved in the present invention in the case of the single-liquid docetaxel-containing pharmaceutical composition for injection.

### Experimental Example 3

### Effects of pH in Inventive Docetaxel Composition

The effects of the solvents on the stability of the injectable docetaxel-containing preparations prepared in Examples 1, 5 to 8, and Comparative examples 2, 7 to 9 were compared and evaluated in the same manner as in Experimental example 1.

TABLE 5

| | Stability at the beginning | | | | Accelerated 1-month storage stability | | | |
|---|---|---|---|---|---|---|---|---|
| | | | | Total related | | | | Total related |
| | | | Content | compounds | | | Content | compounds |
| | Shape | pH | (%) | (%) | Shape | pH | (%) | (%) |
| Ex. 1 | Colorless | 3.81 | 100.5 | 0.705 | Colorless | 3.91 | 100.4 | 1.104 |
| | and clear | | | | and clear | | | |
| Ex. 5 | Colorless | 3.94 | 100.2 | 0.654 | Colorless | 3.95 | 100.2 | 1.210 |
| | and clear | | | | and clear | | | |
| Ex. 6 | Colorless | 4.02 | 100.6 | 0.741 | Colorless | 4.05 | 100.3 | 1.240 |
| | and clear | | | | and clear | | | |
| Ex. 7 | Colorless | 4.47 | 101.5 | 0.716 | Colorless | 4.45 | 100.9 | 1.220 |
| | and clear | | | | and clear | | | |
| Ex. 8 | Colorless | 3.32 | 99.8 | 0.722 | Colorless | 3.30 | 100.5 | 1.100 |
| | and clear | | | | and clear | | | |
| Comp. | Colorless | 6.90 | 101.1 | 0.680 | Light | 6.92 | 42.4 | 30.66 |
| Ex. 2 | and clear | | | | yellow | | | |
| Comp. | Colorless | 6.90 | 101.3 | 0.657 | Light | 6.85 | 40.6 | 32.16 |
| Ex. 7 | and clear | | | | yellow | | | |
| Comp. | Colorless | 6.94 | 100.5 | 0.701 | Light | 6.92 | 39.2 | 35.52 |
| Ex. 8 | and clear | | | | yellow | | | |
| Comp. | Colorless | 5.94 | 100.8 | 0.710 | Colorless | 5.90 | 75.6 | 18.21 |
| Ex. 9 | and clear | | | | and clear | | | |

As seen from the evaluation results of the stabilities of the docetaxel-containing preparations prepared in Examples 1, 5 to 8, and Comparative examples 2, 7 to 9, it was revealed that the compositions whose pH is adjusted with acid to 5 or less have very excellent stability at the beginning and under the accelerated 1-month storage condition, but their shapes, contents and total related compounds are changed to an acute extent when the pH of the compositions is measured to be high at the absence of acid or at a small amount of acid.

Therefore, it was seen that to adjust the pH of the composition to 5 or less is an important factor to secure the stability of the final docetaxel-containing composition in the present invention in the case of the single-liquid docetaxel-containing pharmaceutical composition for injection.

### Experimental example 4

### Evaluation of Dilution Stability in Inventive Docetaxel Compositions

In order to administer the commercially available Taxotere® as the docetaxel-containing injection liquid into human bodies (identical to Comparative example 2 of the present invention), the experiment was carried out based on the following usage and dosage: The Taxotere® was first diluted with 13% (w/w) ethanol dilute solution in the injectable solution, and finally diluted with 0.9% physiological saline or 5% glucose solution to obtain a solution in which docetaxel is present in a concentration of at most 0.74 mg/ml. Then the solution was instilled for 1 hour within 4 hours of the preparation of the solution. Therefore, the stabilities of the preparations according to the present invention should be secured for at least 4 hours without any problems such as the precipitation or eduction for at most 4 hours after the dilution with physiological saline.

1. Evaluation of Dilution Stability: Mixing Degree

In this experiment, the preparations prepared in Examples of the present invention whose stabilities were secured under the accelerated storage condition were compared to the control Taxotere® (Comparative example 2) and the preparations of Comparative examples 3 to 6 so as to evaluate their stabilities. The results are listed in the following Table 6. Levels of the mixing degree as measured during the dilution were set to five grades: very good (5), good (4), mean (3), poor (2) and very poor (1). The grades “very good,” “good,” “mean,” “poor,” and “very poor” refer to the time when a solution is homogeneously mixed with a dilute solution within 30 seconds, 60 seconds, 2 minutes, 3 to 5 minutes and at least 5 minutes, respectively.

TABLE 6
Dilution stability - Mixing Degree

| | Mixing degree |
|---|---|
| Example 1 | 5 |
| Example 3 | 4 |
| Example 5 | 5 |
| Example 6 | 5 |
| Example 7 | 5 |
| Example 8 | 5 |
| Comp. Ex. 2 + | 2 |
| 1^{st} dilute sol. | |
| Comp. Ex. 3 | 3 |
| Comp. Ex. 4 | 3 |
| Comp. Ex. 5 | 5 |
| Comp. Ex. 6 | 5 |

As listed in Table 6, it was revealed that the preparations prepared in Examples of the present invention show their excellent mixing degrees since they are completely diluted within 1 minute, and it does not take additional time to get rid of bubbles caused by the mixing since there is no 1^{st} dilution step. However, the Taxotere® (Comparative example 2) was measured to have a poor mixing degree (Grade 2) even when it is first diluted with a dilute solution, and should be kept for 5 minutes so as to get rid of bubbles caused by the mixing. Also, it was seen that the preparations prepared in Comparative examples 3 and 4 were measured to have a mean mixing degree (Grade 3) in the evaluation of the dilution stability/mixing degree, which indicates that these problems are caused by the mixing. In the case of Comparative examples 5 and 6, it was seen that the preparations are evaluated to have excellent mixing degree, but have a problem in the dilution stability for measuring the precipitation degree.

2. Evaluation of Dilution Stability: Precipitation Degree

Also in this experiment, the preparations of Examples whose stabilities were secured under the accelerated storage condition were compared to the control Taxotere® (Comparative example 2) so as to evaluate their stabilities. The results are listed in the following Table 7. The precipitation degree of the final composition to be administered was set to six grades: very high (5), high (4), mean (3), low (2), very low (1) and nearly naught (0), depending on the amount of precipitate.

TABLE 7
Dilution stability - Precipitation

| | Time | | | | |
|---|---|---|---|---|---|
| | Beginning | 1 hr | 2 hrs | 4 hrs | 6 hrs |
| Ex. 1 + Perfusion liq. | 0 | 0 | 0 | 0 | 1 |
| Ex. 2 + Perfusion liq. | 0 | 0 | 0 | 0 | 1 |
| Ex. 5 + Perfusion liq. | 0 | 0 | 0 | 0 | 1 |
| Ex. 6 + Perfusion liq. | 0 | 0 | 0 | 0 | 1 |
| Ex. 7 + Perfusion liq. | 0 | 0 | 0 | 0 | 1 |
| Ex. 8 + Perfusion liq. | 0 | 0 | 0 | 0 | 1 |
| Comp. Ex. 2 + | 0 | 0 | 1 | 1 | 3 |
| 1^{st} dilute sol. + Perfusion liq. | | | | | |
| Comp. Ex. 3 + Perfusion liq. | 0 | 0 | 0 | 1 | 2 |
| Comp. Ex. 4 + Perfusion liq. | 0 | 0 | 0 | 1 | 2 |
| Comp. Ex. 5 + Perfusion liq. | 0 | 0 | 0 | 1 | 2 |
| Camp. Ex. 6 + Perfusion liq. | 0 | 0 | 0 | 2 | 2 |

From the comparison results of the precipitation degrees with time, it was seen that the commercially available Taxotere® (a final composition for administration of Comparative example 2) starts to precipitate after 2 hours and increasingly precipitate with time. Also, it was revealed that the preparations of Comparative examples have problems in the evaluation of dilution stability/precipitation degree.

As listed in Table 7, it is, however, seen that the final composition diluted with the perfusion liquid in Examples of the present invention show more excellent dilution stabilities than the compositions prepared in Comparative examples 2 to 6.

3. Evaluation of Dilution Stability: Changes in Content

Also in this experiment, the preparations of Examples whose stabilities were secured under the accelerated storage condition were compared to the control Taxotere® (Comparative example 2) so as to evaluate their stabilities. The results are listed in the following Table 8. The changes in the contents with time were analyzed using HPLC.

TABLE 8
Dilution stability - Content

| | Time | | | | |
|---|---|---|---|---|---|
| | Beginning | 1 hr | 2 hrs | 4 hrs | 6 hrs |
| Ex. 1 + Perfusion liq. | 100.0 | 100.6 | 99.2 | 99.2 | 97.3 |
| Ex. 3 + Perfusion liq. | 100.0 | 99.4 | 100.0 | 100.0 | 95.4 |
| Ex. 5 + Perfusion liq. | 100.0 | 100.0 | 100.2 | 100.3 | 97.2 |
| Ex. 6 + Perfusion liq. | 100.0 | 99.7 | 100.2 | 98.7 | 95.5 |
| Ex. 7 + Perfusion liq. | 100.0 | 99.8 | 98.1 | 99.8 | 96.6 |
| Ex. 8 + Perfusion liq. | 100.0 | 100.3 | 99.6 | 99.2 | 96.4 |
| Comp. Ex. 1 + | 100.0 | 100.1 | 94.8 | 92.5 | 87.5 |
| 1^{st} Dilute sol. + Perfusion liq. | | | | | |
| Comp. Ex. 3 + Perfusion liq. | 100.0 | 97.9 | 97.6 | 97.9 | 96.0 |
| Comp. Ex. 4 + Perfusion liq. | 100.0 | 101.6 | 99.0 | 97.8 | 93.2 |
| Comp. Ex. 5 + Perfusion liq. | 100.0 | 99.8 | 100.2 | 96.5 | 91.5 |
| Comp. Ex. 6 + Perfusion liq. | 100.0 | 100.3 | 99.5 | 93.4 | 92.0 |

From the measurement results of the contents with time, it was seen that the dilution stabilities of the preparations of Examples are similar to those as evaluated according to the precipitation degree as listed in Table 7.

From the results of Table 8, it was revealed that the preparations of Comparative examples including the commercially available Taxotere® (a final composition for administration of Comparative example 2) are decreased in content to less than 98% after 4 hours and decreased in content to 87% to 96% after 6 hours. As listed in Table 8, it was, however, seen that the final compositions diluted with the perfusion liquid in Examples of the present invention are highly stable without any of the decrease in content since their contents are measured to be at least 95% after 6 hours.

As a result, the preparations prepared in all Examples of the present invention shows their high contents and good stabilities with regard to the related compounds even when they are diluted with perfusion liquid. On the other hand, the preparation prepared in Comparative example 2 has very low stability in the dilution stability evaluation with regard to the mixing degree, precipitation degree and contents, and the preparations prepared in Comparative examples 3 and 4 show their low loss in content in the dilution stability evaluation, but are negative in the mixing degree and precipitation evaluations. The preparations prepared in Comparative example 5 and 6 have a good mixing degree, but are unstable with the precipitation and the loss in content in the dilution stability evaluation. Also, the preparation prepared in Comparative example 2 shows its low content and negative stability with regard to the related compounds, and the other preparations prepared in Comparative examples 3 to 6 also show their low contents and less stability with regard to the related compounds, compared to those of Examples.

Of all things, since the commercially available Taxotere® (Comparative example 2) undergoes a two-step dilution by primarily diluting Taxotere® with a dilute solution and diluting Taxotere® with 0.9% physiological saline again, the process time is long, the loss of drugs may be caused by mistake during the dilution procedure, and it may be difficult to administer an exact amount of the drugs.

However, it was revealed that the preparations according to the present invention is easy to be used since they are diluted once with 0.9% physiological saline and suitable to administer an exact amount of the docetaxel, and have excellent dilution stabilities, compared to those of the commercially available Taxotere® (Comparative example 2) and the other Comparative examples.

1. A liquid pharmaceutical composition for injection containing docetaxel, comprising:
(A) docetaxel and pharmaceutically acceptable salts thereof;
(B) a surfactant selected from the group consisting of polysorbate, polyoxyethylene glycol ester and polyoxyethylene castor oil derivatives;
(C) a solvent comprising anhydrous ethanol in a concentration range of 100 to 800 mg/ml in an injectable solution, and
(D) a pH adjuster of an amount suitable for adjusting the pH of the liquid composition to 5 or less.

2. The single-liquid docetaxel-containing injectable pharmaceutical composition according to claim 1, wherein the pH of the composition is in a range of 3.0 to 5.0.

3. The single-liquid docetaxel-containing injectable pharmaceutical composition according to claim 1, comprising docetaxel, polysorbate, anhydrous ethanol, and citric acid in an amount suitable for adjusting the pH of the composition to 5 or less.
